(19) United States
(12) Patent Application Publication
(10) Pub. No.: US 2023/0081381 A1
(43) Pub. Date: Mar. 16, 2023

(54) METHODS TO COMBINE FIRST AND SECOND STRAND cDNA SYNTHESIS FOR SPATIAL ANALYSIS

(71) Applicant: 10x Genomics, Inc., Pleasanton, CA (US)

(72) Inventor: Jennifer Chew, Pleasanton, CA (US)

(21) Appl. No.: 17/801,078

(22) PCT Filed: Feb. 19, 2021
(86) PCT No.: PCT/US2021/018816

§ 371 Date: Aug. 19, 2022

Related U.S. Application Data
(60) Provisional application No. 62/979,160, filed on Feb. 20, 2020.

(51) Int. Cl. C12Q 1/6874

(52) U.S. Cl. CPC C12Q 1/6874; C12Q 2600/16; C12Q 1/6855

(57) ABSTRACT

Provided herein are methods of identifying the spatial location of a nucleic acid in a biological sample. In some embodiments, the methods employ a template switching oligonucleotide. In some embodiments, the methods extend the capture probe to create a complementary DNA (cDNA) molecule of a capture analyte and produce second strand in one reaction.

### CROSS-REFERENCE TO RELATED APPLICATION

This application claims priority to U.S. Provisional Patent Application No. 62/979,160, filed Feb. 20, 2020. The contents of this application are incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

### SUMMARY

Provided herein are methods of determining abundance and location of an analyte in a biological sample comprising: (a) contacting a biological sample with a substrate, wherein the substrate comprises a plurality of capture probes attached to the surface of the substrate, and wherein a capture probe of the plurality of capture probes comprises a spatial barcode and a capture domain; (b) hybridizing the analyte to the capture probe; (c) in a single reaction, generating a complementary DNA (cDNA) molecule of the analyte by reverse transcription and performing second strand synthesis of the cDNA molecule by contacting the analyte with a composition comprising a reverse transcription enzyme and a strand-displacing polymerase; and (d) determining (i) all or a portion of the sequence of the spatial barcode or the complement thereof, and (ii) all or a portion of the sequence of the analyte, or a complement thereof; and using the determined sequences of (i) and (ii) to identify the abundance and location of the analyte in the biological sample. In some instances, the composition further comprises a buffer and one or more reagents selected from a plurality of dNTPs, a plurality of template switching oligonucleotides (TSOs), and a plurality of sequences comprising a reverse complement of the TSO (rcTSO).

In some instances, also provided herein is a method of processing an analyte from a biological sample, comprising: (a) hybridizing the analyte from the biological sample to a capture probe, wherein the capture probe comprises a capture domain and a spatial barcode; (b) contacting the analyte with a composition comprising: (i) a buffer; (ii) one or more enzymes selected from a reverse transcription enzyme and a strand-displacing polymerase; and (iii) one or more reagents selected from a plurality of dNTPs, a plurality of template switching oligonucleotides (TSOs), and a plurality of sequences complementary to the TSOs; and (c) performing reverse transcription and second strand synthesis in one reaction.

Also provided herein are methods for identifying abundance and location of an analyte in a biological sample comprising: (a) contacting a biological sample with a substrate, wherein the substrate comprises a plurality of capture probes attached to the surface of the substrate, and wherein a capture probe of the plurality of capture probes comprises a spatial barcode and a capture domain; (b) hybridizing the analyte to the capture probe; (c) contacting the analyte with a composition comprising a reverse transcription enzyme and a strand-displacing polymerase, thereby performing reverse transcription and second strand synthesis in a single reaction; and (d) determining (i) all or a portion of the sequence of the spatial barcode or the complement thereof, and (ii) all or a portion of the sequence of the analyte, or a complement thereof; and using the determined sequences of (i) and (ii) to identify the abundance and location of the analyte in the biological sample.

Also provided herein are methods for processing a nucleic acid from a biological sample, comprising: (a) providing a nucleic acid from a biological sample bound to a capture probe, wherein the capture probe comprises a capture domain and a spatial barcode; (b) contacting the nucleic acid bound to the capture probe with a composition comprising: a buffer; a first enzyme; a template switching oligonucleotide (TSO); and a TSO primer; and performing reverse transcription and second strand synthesis in one reaction.

Provided herein are methods for processing a nucleic acid from a biological sample, comprising: (a) providing a nucleic acid from a biological sample bound to a capture probe, wherein the capture probe comprises a capture domain and a spatial barcode; (b) contacting the nucleic acid bound to the capture probe with a composition comprising: a buffer; a first enzyme; a second enzyme; a template switch oligonucleotide (TSO); and a TSO primer; and (c) performing reverse transcription and second strand synthesis in one reaction.

Provided herein are methods for processing a nucleic acid from a biological sample, comprising: (a) providing a nucleic acid from a biological sample bound to a capture probe, wherein the capture probe comprises a capture domain and a spatial barcode; (b) contacting the nucleic acid bound to the capture probe with a composition comprising a buffer; a first enzyme; and a template switching oligonucleotide (TSO); and (c) performing reverse transcription of the nucleic acid bound to the capture probe, thereby generating a complementary DNA (cDNA) molecule; (d) contacting the nucleic acid bound to the capture probe with a second composition comprising a second enzyme; and a TSO primer; (e) performing second strand synthesis of the cDNA molecule, wherein the steps (a)-(d) are performed in one reaction.

Provided herein are methods of identifying the spatial location of a nucleic acid in a biological sample, comprising: (a) contacting a biological sample with a plurality of capture probes, wherein a capture probe comprises a capture domain and a spatial barcode; (b) releasing a plurality of nucleic acids from the biological sample, wherein a released nucleic acid is specifically bound by the capture domain; (c) contacting the nucleic acid bound to the capture probe with a composition comprising: a buffer; a first enzyme; a template switching oligonucleotide (TSO); and a TSO primer; and (d) determining, for the nucleic acid specifically bound by the capture domain, (1) all or a portion of a sequence of the spatial barcode, or complement thereof, and (2) all or a portion of a sequence of the nucleic acid, or complement thereof, wherein determining includes performing reverse transcription and second strand synthesis in one reaction, thereby identifying the spatial location of a nucleic acid in a biological sample.

Provided herein are methods of identifying the spatial location of a nucleic acid in a biological sample, comprising: (a) contacting the biological sample with a plurality of capture probes, wherein a capture probe comprises a capture domain and a spatial barcode; (b) releasing a plurality of nucleic acids from the biological sample, wherein a released nucleic acid is specifically bound by the capture domain; (c) contacting the nucleic acid bound by the capture probe with a composition comprising: a buffer; a first enzyme; a second enzyme; a template switching oligonucleotide (TSO); and a TSO primer; and (d) determining, for the nucleic acid specifically bound by the capture domain, (1) all or a portion of the sequence of the spatial barcode, or complement thereof, and (2) all or a portion of the sequence of the nucleic acid, or complement thereof, wherein determining includes performing reverse transcription and second strand synthesis in one reaction, thereby identifying the spatial location of a nucleic acid in a biological sample.

Provided herein are methods of identifying the spatial location of a nucleic acid in a biological sample, comprising: (a) releasing a plurality of nucleic acids from the biological sample, wherein a released nucleic acid is specifically bound by a capture probe comprising a capture domain and a spatial barcode; (b) generating a complementary DNA (cDNA) molecule of the bound nucleic acid by reverse transcription with a composition comprising: a buffer; a first enzyme; and a template switching oligonucleotide (TSO); and (c) performing second strand synthesis of the cDNA molecule by contacting the nucleic acid bound by the capture probe with a second composition comprising a second enzyme; and a TSO primer; (d) determining, for the nucleic acid specifically bound by the capture domain, (1) all or a portion of the sequence of the spatial barcode, or complement thereof, and (2) all or a portion of the sequence of the bound nucleic acid, or complement thereof, wherein determining includes performing second strand synthesis immediately after reverse transcription, thereby identifying the spatial location of a nucleic acid in a biological sample.

In some embodiments are methods wherein reverse transcription comprises (i) coupling the TSO to the 3′ end of the nucleic acid; and (ii) extending the capture probe using the captured nucleic acid as a template, thereby generating a cDNA molecule that is complementary to the nucleic acid and the TSO. In some embodiments, second strand synthesis comprises hybridizing the TSO primer to the reverse complement of the TSO (rcTSO); and extending the TSO primer using the extended capture probe as a template, thereby generating a second strand, wherein the second strand is complementary to all or a portion of the cDNA molecule and all or a portion of the capture probe. In some embodiments, the TSO primer binds the rcTSO of the extended capture probe immediately after reverse transcription. In some embodiments, second strand synthesis is performed immediately after reverse transcription. In some embodiments, the method does not comprise a wash step between reverse transcription and second strand synthesis. In some embodiments, the composition further comprises a TSO blocking moiety, wherein the TSO blocking moiety prohibits rcTSO interacting with the TSO primer and is released from the TSO prior to second strand synthesis.

In some instances, the methods further include releasing the second strand. In some instances, the releasing the second strand comprises physical denaturation or chemical denaturation.

In some instances, the composition comprises a template switching oligonucleotide (TSO). In some instances, the TSO comprises a sequence that hybridizes to the capture probe. In some instances, the TSO is about 10 to 50 nucleotides in length. In some instances, the TSO comprises DNA. In some instances, the TSO comprises a homopolymer guanine sequence that hybridizes to a homopolymer cytosine sequence on the capture probe. In some instances, the TSO comprises a sequence that hybridizes to the capture probe.

In some instances, the reverse transcription comprises: (i) coupling the TSO to a 3′ end of the analyte; and (ii) extending the capture probe using the analyte as a template, thereby generating an extended capture probe comprising a sequence that is complementary to the analyte and the TSO. In some instances, the second strand synthesis comprises: (i) hybridizing the TSO to a sequence comprising a reverse complement of the TSO (rcTSO); and (ii) extending the TSO using the extended capture probe as a template, thereby generating a second strand, wherein the second strand is complementary to all or a portion of the analyte and all or a portion of the capture probe. In some instances, the rcTSO comprises DNA.

In some instances, the TSO binds the rcTSO of the extended capture probe immediately after reverse transcription.

In some instances, second strand synthesis is performed immediately after the reverse transcription.

In some instances, the method does not comprise a wash step between reverse transcription and second strand synthesis.

In some instances, the methods disclosed herein further include ligating the rcTSO to the capture probe using a ligase. In some instances, the ligase is a T4 RNA ligase (Rn12), a splintR ligase, a single stranded DNA ligase, or a T4 DNA ligase. In some instances, the rcTSO comprises a pre-adenylated phosphate group at its 5′ end, and wherein the first probe comprises at least two ribonucleic acid bases at the 3′ end. In some instances, the ligating the rcTSO to the capture probe comprises ligating a 3′ end of the capture probe to a 5′ end of the rcTSO. In some instances, the ligase is selected from the group consisting of: thermostable 5′ AppDNA/RNA Ligase, truncated T4 RNA Ligase 2, truncated T4 RNA Ligase 2 K227Q, truncated T4 RNA Ligase 2 KQ, Chlorella Virus PBCV-1 DNA Ligase, or any combination thereof.

In some embodiments, the first enzyme comprises a reverse transcriptase enzyme. In some embodiments, the first enzyme comprises a reverse transcriptase enzyme comprising one or more of terminal transferase activity, template switching ability, strand displacement ability, or combinations thereof. In some embodiments, the first enzyme comprises a Moloney Murine Leukemia Virus (M-MLV) reverse transcriptase enzyme. In some embodiments, the first enzyme comprises M-MLV reverse transcriptase enzyme 42B. In some embodiments, the TSO comprises a hybridization region and template region. In some embodiments, the template region comprises a primer sequence.

In some embodiments, the composition further comprises a second enzyme. In some embodiments, the second enzyme is a strand-displacing polymerase. In some embodiments, the second enzyme is a phi29 DNA polymerase. In some embodiments, the second enzyme is a Bst DNA polymerase. In some embodiments, the method further comprises releasing the second strand. In some embodiments, releasing the second strand comprises physical denaturation. In some embodiments, releasing the second strand comprises chemical denaturation.

In some embodiments, the method further comprises a plurality of capture probes. In some embodiments, the plurality of capture probes are attached to a substrate. In some embodiments, the substrate is a feature. In some embodiments, the feature a bead. In some embodiments, the capture probe comprises a capture domain, a spatial barcode, and a UMI. In some instances, the capture probe further comprises one or more functional domains, a unique molecular identifier (UMI), a cleavage domain, and combinations thereof.

In some embodiments, the biological sample comprises a tissue section. In some embodiments, the biological sample comprises a formalin-fixed, paraffin-embedded (FFPE) sample, a frozen sample, or a fresh sample. In some embodiments, the biological sample comprises an FFPE sample. In some instances, the FFPE tissue sample is decrosslinked.

In some embodiments, the biological sample is removed from the substrate after hybridization of the analyte to the capture probe. In some embodiments, the biological sample was previously stained. In some embodiments, the biological sample was previously stained using immunofluorescence or immunohistochemistry. In some embodiments, the biological sample was previously stained using hematoxylin and eosin.

In some embodiments, the analyte comprises a nucleic acid. In some embodiments, the nucleic acid is an RNA molecule. In some embodiments, the RNA molecule is an mRNA molecule.

In some embodiments, the methods further include permeabilizing the biological sample with a permeabilization agent. In some embodiments, the permeabilization agent is selected from an organic solvent, a detergent, and an enzyme, or a combination thereof. In some embodiments, the permeabilization agent is selected from the group consisting of: an endopeptidase, a protease sodium dodecyl sulfate (SDS), polyethylene glycol tert-octylphenyl ether, polysorbate 80, and polysorbate 20, N-lauroylsarcosine sodium salt solution, saponin, Triton X-100™, and Tween-20™. In some embodiments, the endopeptidase is pepsin. In some embodiments, the endopeptidase is proteinase K. In some embodiments, the method further comprises, prior to step (a), fixing the biological sample. In some embodiments, the step of fixing the biological sample is performed using one or both of methanol and acetone.

In some embodiments, the determining step comprises amplifying all or part of the analyte hybridized to the capture domain, thereby producing an amplifying product. In some embodiments, an amplifying product comprises (i) all or part of analyte hybridized to the capture domain, or a complement thereof, and (ii) all or a part of the spatial barcode, or a complement thereof. In some embodiments, the determining step comprises sequencing. In some embodiments, the sequencing comprises in situ sequencing, Sanger sequencing methods, next-generation sequencing methods, and nanopore sequencing.

Also disclosed herein are kits. In some instances, the kits include (a) a substrate comprises a plurality of captures probes attached to the surface of the substrate, wherein a capture probe of the plurality of capture probes comprises a spatial barcode and a capture domain; (b) one or more reagents selected from a plurality of dNTPs, a plurality of template switching oligonucleotides (TSOs); a plurality of sequences complementary to the TSOs; (c) one or more enzymes selected from a reverse transcriptase and a polymerase; and (d) instructions for performing any of the methods disclosed herein.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, including mixtures thereof “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A-6C are schematics illustrating how streptavidin cell tags can be utilized in an array-based system to produce a spatially-barcoded cells or cellular contents.

FIG. 7 is a schematic diagram showing an exemplary method combining reverse transcription and second strand synthesis in one reaction.

FIGS. 8A-8H show heat maps of transcripts detected on a spatial array.

FIG. 9 shows summary statistics for mapped and usable reads for each of the plots in FIGS. 8A-8H.

FIG. 10 shows summary statistics for read quality for each of the plots in FIGS. 8A-8H.

FIG. 11 shows median genes per spot for each of the plots in FIGS. 8A-8H.

FIG. 12 shows median UMIs per spot for each of the plots in FIGS. 8A-8H.

FIGS. 13A-13B show heat maps of transcripts detected on a spatial array using a mouse brain tissue sample.

FIGS. 14A-14B show heat maps of transcripts detected on a spatial array using a mouse brain tissue sample.

FIG. 15 shows summary statistics for mapped and usable reads for each of the plots in FIGS. 13A, 13B, 14A, and 14B.

FIG. 16 shows percentage of valid barcodes and fraction reads per spot for each of the plots in FIGS. 13A, 13B, 14A, and 14B.

FIG. 17 shows median genes per spot and median UMIs for each of the plots in FIGS. 13A, 13B, 14A, and 14B.

### DETAILED DESCRIPTION

### I. Introduction

Spatial analysis methods using capture probes and/or analyte capture agents provide information regarding the abundance and location of an analyte (e.g., a nucleic acid or protein). The efficiency of spatial analysis using arrays with capture probes depends, at least in part, on the density of the probes on the array or the density of the analytes captured on the array. That is, on how many capture probes can be printed on the surface of a slide or how many RNA molecules can be captured. Disclosed herein are methods and compositions for increasing the efficiency of spatial analysis by increasing the number of interactions between the capture probe and the analyte. In this way, analyte detection signal is increased, thus increasing the capturing efficiency, sensitivity, and the resolution of detection on the spatial array.

Traditionally, these methods identify a singular molecule at a location. Extending these methods to study interactions between two or more analytes would provide information on the interactions between two or more analytes at a location in a biological sample. Analyte capture agents as provided herein comprises an analyte binding moiety affixed to an oligonucleotide. The oligonucleotide comprises a sequence that uniquely identifies the analyte and moiety. Further, nearby oligonucleotides affixed to a different moiety in a nearby location can be ligated to the first oligonucleotide and then can be detected using the spatial methods described herein. The methods disclosed herein thus provide the ability to study the interaction between two or more analytes in a biological sample.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample. In some instance, the capture domain is designed to detect one or more specific analytes of interest. For example, a capture domain can be designed so that it comprises a sequence that is complementary or substantially complementary to one analyte of interest. Thus, the presence of a single analyte can be detected. Alternatively, the capture domain can be designed so that it comprises a sequence that is complementary or substantially complementary to a conserved region of multiple related analytes. In some instances, the multiple related analytes are analytes that function in the same or similar cellular pathways or that have conserved homology and/or function. The design of the capture probe can be determined based on the intent of the user and can be any sequence that can be used to detect an analyte of interest. In some embodiments, the capture domain sequence can therefore be random, semi-random, defined or combinations thereof, depending on the target analyte(s) of interest.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

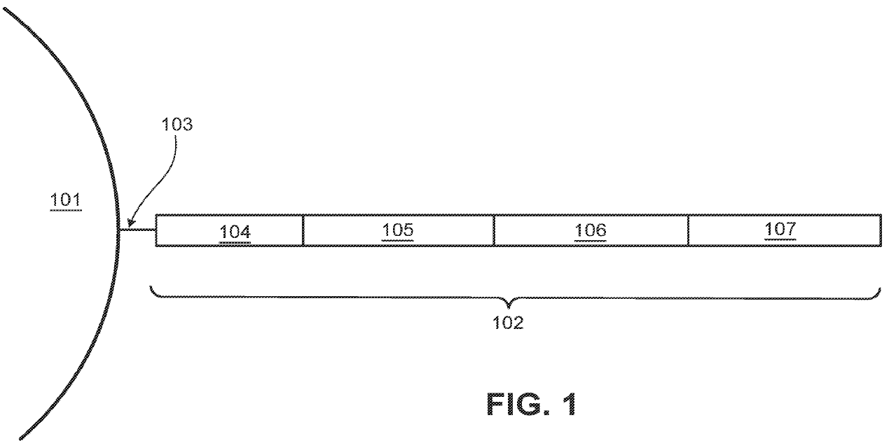

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

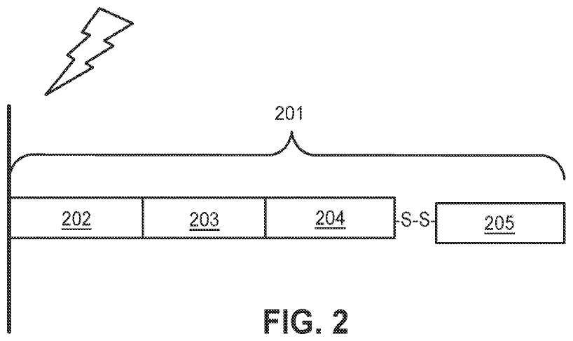

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

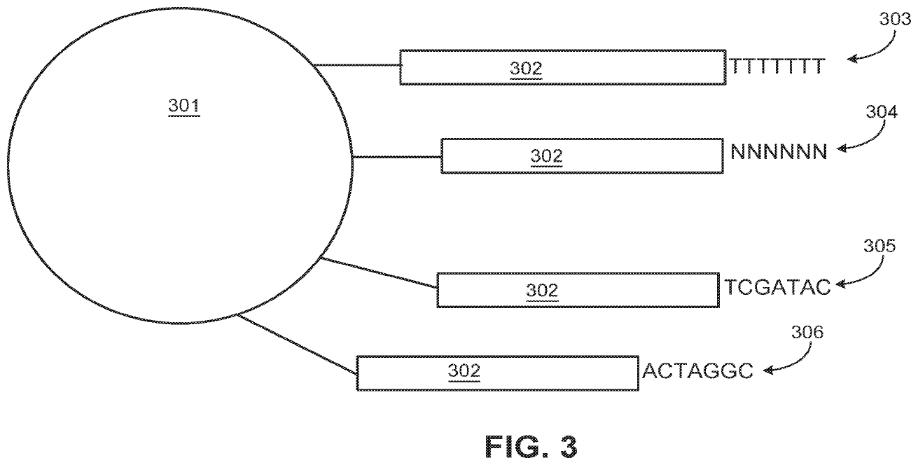

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

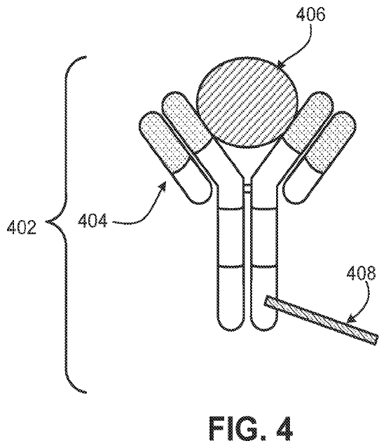

FIG. 4 is a schematic diagram of an exemplary analyte capture agent 402 comprised of an analyte-binding moiety 404 and an analyte-binding moiety barcode domain 408. The exemplary analyte -binding moiety 404 is a molecule capable of binding to an analyte 406 and the analyte capture agent is capable of interacting with a spatially-barcoded capture probe. The analyte -binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte -binding moiety 404 can include a polypeptide and/or an aptamer. The analyte -binding moiety 404 can include an antibody or antibody fragment (e.g., an antigen-binding fragment).

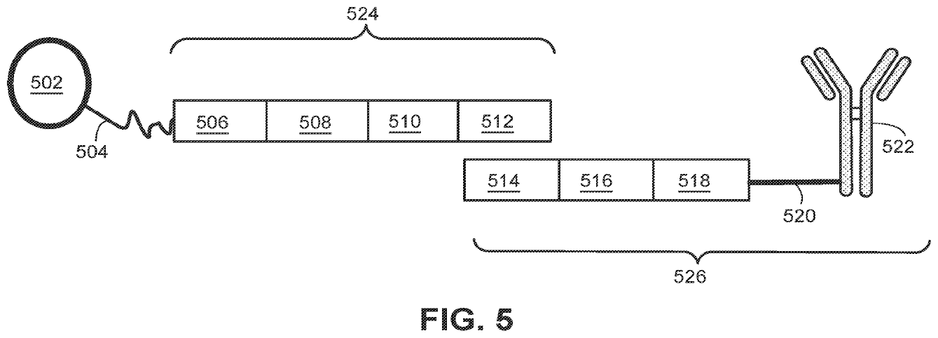

FIG. 5 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

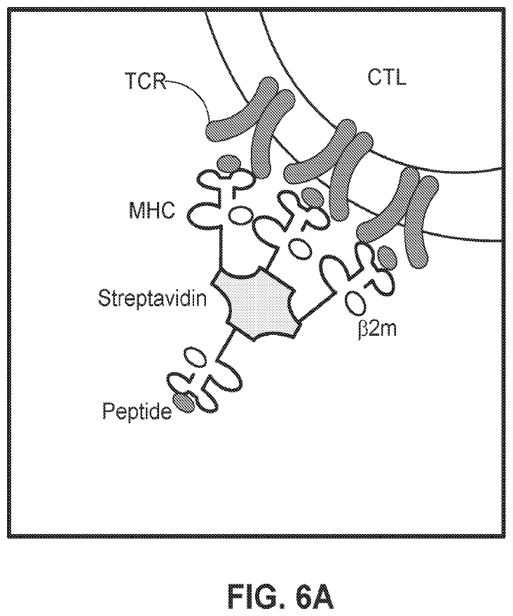

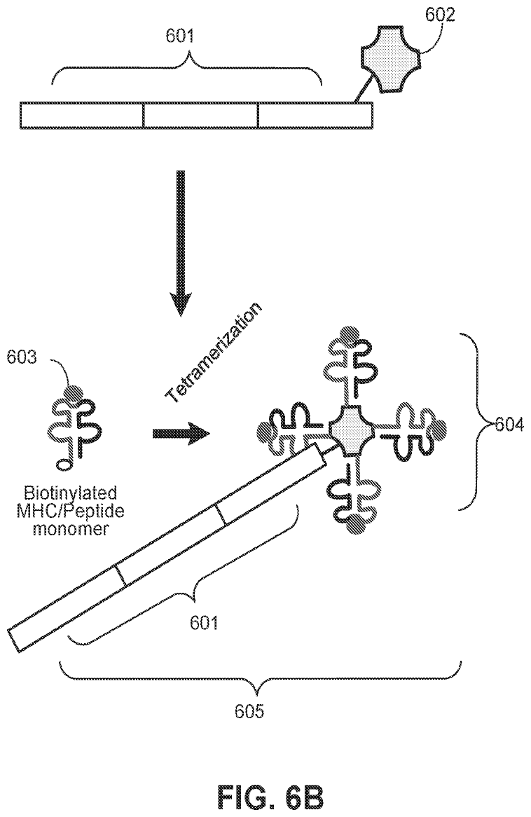

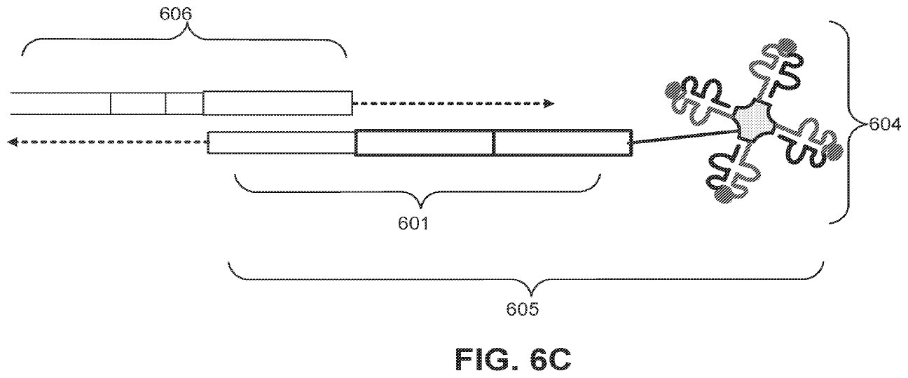

FIGS. 6A, 6B, and 6C are schematics illustrating how streptavidin cell tags can be utilized in an array-based system to produce a spatially-barcoded cell or cellular contents. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a TCR such that the streptavidin binds to a target T-cell via multiple MCH/TCR binding interactions. Multiple interactions synergize and can substantially improve binding affinity. Such improved affinity can improve labelling of T-cells and also reduce the likelihood that labels will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601. The result is a barcoded MHC multimer complex 605. As shown in FIG. 6B, the capture agent barcode domain sequence 601 can identify the MHC as its associated label and also includes optional functional sequences such as sequences for hybridization with other oligonucleotides. As shown in FIG. 6C, one example oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a gel bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of these corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from spatial barcode sequence on the capture agent barcode domain 601 may be used to identify the particular peptide MHC complex 604 bound on the surface of the cell (e.g., when using MHC-peptide libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligation products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease (e.g., RNAse H). The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Additional spatial processes are described in PCT Patent Application Publication No. WO 2020/123320, which is incorporated by reference in its entirety.

II. Processing a Nucleic Acid from a Biological Sample

(a) Introduction

The disclosure provides methods and compositions that enhance the efficiency of spatial detection. Traditionally, a spatially-barcoded, first-strand cDNA is generated via extension of the capture domain of the capture probe using the captured analyte as a template, followed by template-switch chemistry with an enzyme (e.g., a reverse transcriptase). Afterwards, the analyte (e.g., RNA) is denatured from the RNA-DNA hybrids created during the RT reaction, and a second reaction is performed using a DNA polymerase. As disclosed herein, combining one or more steps during reverse transcription and amplification of the analyte results does not affect the downstream analysis (e.g., sequencing) of the analyte. Compared to traditional spatial analysis, the methods disclosed herein are less-time consuming and require less reagents since the first- and second-strand synthesis reactions are combined into a single reaction, without the need to denature, rinse, and add new reagents following reverse transcription to create a second-strand. Further, it has been identified that some enzymes, such as M-MLV, act as both an RNA- and DNA-dependent DNA polymerase and have been shown to have some RNA displacement activity. See e.g., Kelleher et al., J Biol Chem, 1998: 273(16):9976-86; and Tzanetakis et al., J Virol Methods, 2005; 124(1-2):73-7, each of which is herein incorporated by reference in its entirety. The methods and compositions provided herein harness the strand-displacing properties of an enzyme such as M-MLV along with template switch chemistry to generate barcoded second strand cDNA immediately after the first strand is created. By doing so, time, reagents and cost of spatial reactions are maximized.

Thus, in some instances, the methods disclosed herein relate to methods of identifying abundance and location of an analyte in a biological sample comprising: (a) contacting a biological sample with a substrate, wherein the substrate comprises a plurality of capture probes attached to the surface of the substrate, and wherein a capture probe of the plurality of capture probes comprises a spatial barcode and a capture domain; (b) hybridizing the analyte to the capture probe; (c) contacting the analyte with a composition comprising a reverse transcription enzyme and a strand-displacing polymerase, thereby performing reverse transcription and second strand synthesis simultaneously in a reaction; and (d) determining (i) all or a portion of the sequence of the spatial barcode or the complement thereof, and (ii) all or a portion of the sequence of the analyte, or a complement thereof; and using the determined sequences of (i) and (ii) to identify the abundance and location of the analyte in the biological sample.

In addition, this disclosure feature methods for processing a nucleic acid from a biological sample and methods for identifying the spatial location of a nucleic acid in a biological sample. Provided herein are methods for processing a nucleic acid where reverse transcription and second strand synthesis are performed in one reaction. For example, a method for processing a nucleic acid includes reverse transcription immediately followed (e.g., no washing step) by second strand synthesis, or for all intents and purposes simultaneously when all reagents are combined in one reaction. In some embodiments, a method for processing a nucleic acid where reverse transcription and second strand synthesis are performed in one reaction includes a first enzyme, a template switch oligonucleotide (TSO), and a TSO primer. In some embodiments, a method for processing a nucleic acid where reverse transcription and second strand synthesis are performed in one reaction includes a first enzyme, a second enzyme, a TSO, and a TSO primer. Also provided herein are methods of identifying a spatial location of a nucleic acid in a biological sample where reverse transcription and second strand synthesis are performed in one reaction. In some embodiments of identifying a spatial location of a nucleic acid in a biological sample where reverse transcription and second strand synthesis are performed in one reaction, the methods includes a first enzyme, a TSO, and a TSO primer. In some embodiments of identifying a spatial location of a nucleic acid in a biological sample where reverse transcription and second strand synthesis are performed in one reaction, the methods include a first enzyme, a second enzyme, a TSO, and a TSO primer.

The methods provided herein avoid the need for separate reactions (e.g., additional processing) for first strand complementary DNA (cDNA) synthesis and second strand synthesis. This reduces the reaction time by combining two reactions into one and the amount of reagents needed for denaturation, rinsing, and addition of supplemental reagents thereby creating time and cost efficiencies while maintaining sensitivity of the spatial assay.

Additional embodiments, including compositions and kits, of the disclosure are provided herein.

(b) Methods of Spatial Analysis of an Analyte

In some embodiments, the method for analysis of an analyte (e.g., a nucleic acid) from a biological sample includes performing reverse transcription and second strand synthesis in one reaction. In some embodiments, the method for processing a nucleic acid from a biological sample where reverse transcription and second strand synthesis are performed in one reaction includes providing a nucleic acid from a biological sample bound to a capture probe, where the capture probe includes a capture domain and a spatial barcode; contacting the nucleic acid bound to the capture probe with a composition including: a buffer; a first enzyme; a template switching oligonucleotide (TSO); and a TSO primer; and performing reverse transcription and second strand synthesis. In some embodiments, the method for processing a nucleic acid from a biological sample where reverse transcription and second strand synthesis are performed in one reaction includes providing a nucleic acid from a biological sample bound to a capture probe, where the capture probe includes a capture domain and a spatial barcode; contacting the nucleic acid bound to the capture probe with a composition including: a buffer; a first enzyme; a second enzyme; a template switch oligonucleotide (TSO); and a TSO primer; and performing reverse transcription and second strand synthesis in one reaction. In some embodiments, the method for processing a nucleic acid from a biological sample where reverse transcription and second strand synthesis are performed in one reaction includes: (a) providing a nucleic acid from a biological sample bound to a capture probe, where the capture probe includes a capture domain and a spatial barcode; (b) contacting the nucleic acid bound to the capture probe with a composition including: a buffer; a first enzyme; and a template switching oligonucleotide (or oligo) (TSO); and (c) performing reverse transcription of the nucleic acid bound to the capture probe, thereby generating a complementary DNA (cDNA) molecule; (d) contacting the nucleic acid bound to the capture probe with a second composition including: a second enzyme; and a TSO primer; (e) performing second strand synthesis of the cDNA molecule, where the steps (a)-(d) are performed in one reaction.

In some embodiments of any of the methods of processing a nucleic acid from a biological sample where reverse transcription and second strand synthesis are performed in one reaction, the TSO primer binds a sequence having a reverse complement of the TSO sequence (herein referred to as an “rcTSO”) of the extended capture probe immediately after reverse transcription. In some instances, the rcTSO hybridizes to the TSO, bringing it in proximity to the capture probe. Once in proximity, the capture probe and the rcTSO are ligated.

In some embodiments, the second strand synthesis is performed immediately after reverse transcription. In some embodiments, the method does not comprise a wash step between reverse transcription and second strand synthesis.

In some embodiments of any of the methods of identifying the spatial location of a nucleic acid in a biological sample where reverse transcription and second strand synthesis are performed in one reaction, the method includes adding an additional volume of any of the compositions described herein to the reaction. In some embodiments, the method includes adding a second additional volume of any of the compositions described herein to the reaction. In some embodiments of any of the method where one or more additional volumes of any of the compositions described herein are added to the reaction, the compositions are added without performing a wash step. In some embodiments, the additional volume of compositions added to the reaction includes the first enzyme. In some embodiments, the additional volume of compositions added to the reaction includes the first enzyme and a TSO. In some embodiments, the additional volume of compositions added to the reaction includes the first enzyme, a TSO, and a TSO primer. In some embodiments, the additional volume of compositions added to the reaction includes the first enzyme, a second enzyme, a TSO, and a TSO primer. In some embodiments, the additional volume of compositions added to the reaction includes the first enzyme and the second enzyme. In some embodiments, the additional volume of compositions added to the reaction includes the second enzyme. In some embodiments, the additional volume of compositions added to the reaction includes the second enzyme and a TSO. In some embodiments, the additional volume of compositions added to the reaction includes the second enzyme, a TSO, and a TSO primer. In some embodiments, the additional volume of compositions added to the reaction includes the second enzyme and a TSO primer. In some embodiments, the additional volume of composition added to the reaction includes a first buffer and/or a second buffer.

In some embodiments, a method of identifying the spatial location of a nucleic acid in a biological sample includes a method where reverse transcription and second strand synthesis are performed in one reaction. In some embodiments, the method of identifying the spatial location of a nucleic acid in a biological sample where reverse transcription and second strand synthesis are performed in one reaction includes: contacting a biological sample with a plurality of capture probes, where a capture probe includes a capture domain and a spatial barcode; releasing a plurality of nucleic acids from the biological sample, where a released nucleic acid is specifically bound by the capture domain, contacting the nucleic acid bound to the capture probe with a composition including: a buffer; a first enzyme; a template switching oligonucleotide (TSO), and a TSO primer, and determining, for the nucleic acid specifically bound by the capture domain, all or a portion of a sequence of the spatial barcode, or complement thereof, and all or a portion of a sequence of the nucleic acid, or complement thereof, where determining includes performing reverse transcription and second strand synthesis in one reaction, thereby identifying the spatial location of a nucleic acid in a biological sample.

In some embodiments, the method of identifying the spatial location of a nucleic acid in a biological sample where reverse transcription and second strand synthesis are performed in one reaction includes: contacting the biological sample with a plurality of capture probes, where a capture probe includes a capture domain and a spatial barcode, releasing a plurality of nucleic acids from the biological sample, where a released nucleic acid is specifically bound by the capture domain, contacting the nucleic acid bound by the capture probe with a composition including: a buffer; a first enzyme; a second enzyme, a template switching oligonucleotide (TSO), and a TSO primer; and determining, for the nucleic acid specifically bound by the capture domain, all or a portion of the sequence of the spatial barcode, or complement thereof, and all or a portion of the sequence of the nucleic acid, or complement thereof, where determining includes performing reverse transcription and second strand synthesis in one reaction, thereby identifying the spatial location of a nucleic acid in a biological sample.

In some embodiments, the method of identifying the spatial location of a nucleic acid in a biological sample where reverse transcription and second strand synthesis are performed in one reaction includes: releasing a plurality of nucleic acids from the biological sample, where a released nucleic acid is specifically bound by a capture probe including a capture domain and a spatial barcode, generating a complementary DNA (cDNA) molecule of the bound nucleic acid by reverse transcription with a composition including: a buffer, a first enzyme, and a template switching oligonucleotide (TSO), and performing second strand synthesis of the cDNA molecule by contacting the nucleic acid bound by the capture probe with a second composition including: a second enzyme, and a TSO primer, determining, for the nucleic acid specifically bound by the capture domain, all or a portion of the sequence of the spatial barcode, or complement thereof, and all or a portion of the sequence of the bound nucleic acid, or complement thereof, where determining includes performing second strand synthesis immediately after reverse transcription, thereby identifying the spatial location of a nucleic acid in a biological sample.

In some embodiments of any of the methods of identifying the spatial location of a nucleic acid in a biological sample where reverse transcription and second strand synthesis are performed in one reaction, the TSO primer binds the rcTSO of the extended capture probe immediately after reverse transcription. In some embodiments, the second strand synthesis is performed immediately after reverse transcription. In some embodiments, the method does not include a wash step between reverse transcription and second strand synthesis.

In some embodiments of any of the methods of identifying the spatial location of a nucleic acid in a biological sample where reverse transcription and second strand synthesis are performed in one reaction, the method includes adding an additional volume of any of the compositions described herein to the reaction. In some embodiments, the method includes adding a second additional volume of any of the compositions described herein to the reaction. In some embodiments of any of the method where one or more additional volumes of any of the compositions described herein are added to the reaction, the compositions are added without performed a wash step. In some embodiments, the additional volume of compositions added to the reaction includes the first enzyme. In some embodiments, the additional volume of compositions added to the reaction includes the first enzyme and a TSO. In some embodiments, the additional volume of compositions added to the reaction includes the first enzyme, a TSO, and a TSO primer. In some embodiments, the additional volume of compositions added to the reaction includes the first enzyme, a second enzyme, a TSO, and a TSO primer. In some embodiments, the additional volume of compositions added to the reaction includes the first enzyme and the second enzyme. In some embodiments, the additional volume of compositions added to the reaction includes the second enzyme. In some embodiments, the additional volume of compositions added to the reaction includes the second enzyme and a TSO. In some embodiments, the additional volume of compositions added to the reaction includes the second enzyme, a TSO, and a TSO primer. In some embodiments, the additional volume of compositions added to the reaction includes the second enzyme and a TSO primer. In some embodiments, the additional volume of composition added to the reaction includes a first buffer and/or a second buffer.

In some instances, the reverse transcription (RT) reaction of the single step reaction disclosed herein is performed at about 53° C. In some instances, the RT reaction of the single step reaction disclosed herein is performed at about 45° to 65° C. In some instances, the reverse transcription reaction of the single step reaction disclosed herein is performed for about 5 minutes of the single step reaction. In some instances, the reverse transcription reaction of the single step reaction disclosed herein is performed for about 10 minutes of the single step reaction. In some instances, the reverse transcription reaction of the single step is performed for about 5 minutes to about 2 hours (e.g., for about 5, 10, 15, 20, 25 30, 40, 50, 60, 70, 80, 90, 100, 110, or 120 minutes) of the single step reaction. In some instances, the concentration of the reverse transcriptase is about 0.1 U/μl to about 2.0 U/μ1 (e.g., about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 U/μl). It is appreciated that one skilled in the art could readily determine the temperature and time of the RT reaction and the concentration of the RT.

In some instances, the second strand synthesis reaction of the single step reaction disclosed herein is performed at about 65° C. In some instances, the second strand synthesis reaction of the single step reaction disclosed herein is performed at about 50° C. to 75° C. In some instances, the second strand synthesis reaction of the single step reaction disclosed herein is performed for about 5 minutes of the single step reaction. In some instances, the second strand synthesis reaction of the single step reaction disclosed herein is performed for about 10 minutes of the single step reaction. In some instances, the second strand synthesis reaction of the single step is performed for about 5 minutes to about 2 hours (e.g., for about 5, 10, 15, 20, 25 30, 40, 50, 60, 70, 80, 90, 100, 110, or 120 minutes) of the single step reaction. In some instances, the concentration of the second strand synthesis polymerase is about 0.1 U/μl to about 2.0 U/μl (e.g., about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 U/μl). It is appreciated that one skilled in the art could readily determine the temperature and time of the second strand synthesis reaction and the concentration of the polymerase.

In some embodiments, after contacting a biological sample with the capture probes, a removal step can optionally be performed to remove all or a portion of the biological sample from the surface of a slide. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, provided herein are methods for spatially detecting an analyte (e.g., detecting the location of an analyte, e.g., a biological analyte) from a biological sample (e.g., present in a biological sample), the method comprising: (a) optionally staining and/or imaging a biological sample on a substrate; (b) permeabilizing (e.g., providing a solution comprising a permeabilization reagent to) the biological sample on the substrate; (c) contacting the biological sample with the capture probes as described herein, wherein a capture probe of the plurality captures the biological analyte; and (d) analyzing the captured biological analyte, thereby spatially detecting the biological analyte.

In some embodiments, a full-length DNA (e.g., cDNA) molecule is generated. In some embodiments, a “full-length” DNA molecule refers to the whole of the captured nucleic acid molecule. However, if a nucleic acid (e.g., RNA) was partially degraded in the tissue sample, then the captured nucleic acid molecules will not be the same length as the original RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Lucigen, Middleton, Wis.). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9oN) DNA ligase (9oN™ DNA ligase, New England Biolabs), Ampligase™ (available from Lucigen, Middleton, Wis.), and SplintR (available from New England Biolabs, Ipswich, Mass.). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some embodiments, double-stranded extended capture probes are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the amplification reaction incorporates an affinity group onto the extended capture probe (e.g., RNA-cDNA hybrid) using a primer including the affinity group. In some embodiments, the primer includes an affinity group and the extended capture probes includes the affinity group. The affinity group can correspond to any of the affinity groups described previously.

In some embodiments, the extended capture probes including the affinity group can be coupled to a substrate specific for the affinity group. In some embodiments, the substrate can include an antibody or antibody fragment. In some embodiments, the substrate includes avidin or streptavidin and the affinity group includes biotin. In some embodiments, the substrate includes maltose and the affinity group includes maltose-binding protein. In some embodiments, the substrate includes maltose-binding protein and the affinity group includes maltose. In some embodiments, amplifying the extended capture probes can function to release the extended probes from the surface of the substrate, insofar as copies of the extended probes are not immobilized on the substrate.

In some embodiments, the extended capture probe or complement or amplicon thereof is released from the biological sample. The step of releasing the extended capture probe or complement or amplicon thereof from the biological sample can be achieved in a number of ways. In some embodiments, an extended capture probe or a complement thereof is released from the array by nucleic acid cleavage and/or by denaturation (e.g., by heating to denature a double-stranded molecule).

In some embodiments, the extended capture probe or complement or amplicon thereof is released from the biological sample by physical means. In some embodiments, the extended capture probe is released by applying a heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended capture probe from the biological sample. In some embodiments, where the extended capture probe includes a cleavage domain, the extended capture probe is released from the biological sample by cleavage.

In some embodiments, probes complementary to the extended capture probe can be contacted with the substrate. In some embodiments, the biological sample can be in contact with the substrate when the probes are contacted with the substrate. In some embodiments, the probes can be labeled with a detectable label (e.g., any of the detectable labels described herein). In some embodiments, probes that do not specially bind (e.g., hybridize) to an extended capture probe can be washed away. In some embodiments, probes complementary to the extended capture probe can be detected on the substrate (e.g., imaging, any of the detection methods described herein).

In some embodiments, probes complementary to an extended capture probe can be about 4 nucleotides to about 100 nucleotides long. In some embodiments, probes (e.g., detectable probes) complementary to an extended capture probe can be about 10 nucleotides to about 90 nucleotides long. In some embodiments, probes (e.g., detectable probes) complementary to an extended capture probe can be about 20 nucleotides to about 80 nucleotides long. In some embodiments, probes (e.g., detectable probes) complementary to an extended capture probe can be about 30 nucleotides to about 60 nucleotides long. In some embodiments, probes (e.g., detectable probes) complementary to an extended capture probe can be about 40 nucleotides to about 50 nucleotides long. In some embodiments, probes (e.g., detectable probes) complementary to an extended capture probe can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 nucleotides long.

In some embodiments, the probes can be complementary to a single analyte (e.g., a single gene). In some embodiments, the probes can be complementary to one or more analytes (e.g., analytes in a family of genes). In some embodiments, the probes (e.g., detectable probes) can be for a panel of genes associated with a disease (e.g., cancer, Alzheimer's disease, Parkinson's disease).

In some instances, the ligated probe and capture probe can be amplified or copied, creating a plurality of cDNA molecules. In some embodiments, cDNA can be denatured from the capture probe template and transferred (e.g., to a clean tube) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flowcell (Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

In some embodiments, where a sample is barcoded directly via hybridization with capture probes or analyte capture agents hybridized, bound, or associated with either the cell surface, or introduced into the cell, as described above, sequencing can be performed on the intact sample.

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

(c) Enzymes

In some embodiments, the methods and compositions include one or more enzymes used in the same reaction. In some instances, the one or more enzymes (also referred to herein as a first enzyme, a second enzyme, and so one) include a reverse transcriptase enzyme and a polymerase enzyme. As described above, the concentrations and volumes of the enzymes disclosed herein can be determined by one skilled in the art.

In the instances, the reverse transcriptase enzyme includes one or more of terminal transferase activity, template switching ability, strand displacement ability, or combinations thereof. In some embodiments, the terminal transferase activity of the reverse transcriptase adds untemplated nucleotides to the 3′ end of the cDNA molecule. In some embodiments, the reverse transcriptase adds 1, 2, 3, 4, 5, 6, 7, 8, 9, 10, or more untemplated nucleotides to the 3′ end of the cDNA molecule. In some embodiments the reverse transcriptase comprises a Moloney Murine Leukemia Virus (M-MLV) reverse transcriptase enzyme. In some embodiments the reverse transcriptase enzyme comprises a Moloney Murine Leukemia Virus (M-MLV) reverse transcriptase enzyme and the second enzyme is a Bst DNA polymerase. In some embodiments the reverse transcriptase enzyme comprises a Moloney Murine Leukemia Virus (M-MLV) reverse transcriptase enzyme. In some embodiments, the reverse transcriptase enzyme is M-MLV reverse transcriptase enzyme 42B.

In some instances, the polymerase is a DNA polymerase. In some instances, the polymerase is a Phi29 DNA polymerase. In some embodiments, second strand synthesis is performed by a DNA polymerase selected from the group including, but not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes. In some embodiments, the second strand synthesis is a Phi29 DNA polymerase. In some embodiments, the second strand synthesis is performed by a Bst DNA polymerase.

In some instances, additional enzymes such as RNase H are not utilized. Instead, only the gap between TSO and the original strand of mRNA is used to initiate second strand synthesis.

(d) TSO and TSO Primer

In some embodiments, the method includes contacting the nucleic acid bound to the capture probe with a composition that includes a template switching oligonucleotide (TSO). In some embodiments, a TSO includes a hybridization region (e.g., an untemplated nucleotide region) and a template region (e.g., TSO primer region). In some embodiments, the length of a template switching oligonucleotide can be at least about 1, 2, 10, 20, or 50 nucleotides or longer. In some embodiments, the length of a template switching oligonucleotide can be at most about 2, 10, 20, 50, 100, 150, 200, or 250 nucleotides or longer.

In some embodiments, the template region (e.g., TSO primer region) includes a sequence that is at least partially complementary to the TSO primer. In some embodiments, the template region (e.g., TSO primer region) includes a sequence that is 1, 2, 3, 4, 5, 6, 7, 8, 9, 10, 11, 12, 13, 14, 15, 16, 17, 18, 19, 20, 21, 22, 23, 24, 25, or nucleotides in length. In some embodiments, the hybridization region includes a sequence that is at least partially complementary to the untemplated nucleotides added on to the 3′ end of the extended capture probe. In some embodiments, the hybridization region includes a sequence that is 1, 2, 3, 4, 5, 6, 7, 8, 9, 10, or more nucleotides in length.

In some embodiments, the hybridization region includes a series of guanine (G) bases to complement the overhanging cytosine (C) bases at the 3′ end of a cDNA molecule. In some embodiments, the series of G bases can include 1 G base, 2 G bases, 3 G , 4 G bases, 5 G bases, or more than 5 G bases. In some embodiments, the hybridization region can include at least one base in addition to at least one G base. In other embodiments, the hybridization region can include bases that are not a G base. In some embodiments, the template region (e.g., TSO primer region) and hybridization region are separated by a spacer. In some embodiments, the reverse complement of the TSO (rcTSO) is incorporated at the 3′ end of the cDNA molecule when the TSO binds to the untemplated nucleotides on the cDNA molecule and the reverse transcriptase reverse transcribes the TSO.

In some embodiments, the method includes contacting the nucleic acid bound to the capture probe with a composition that includes a TSO primer. In some embodiments, the TSO primer includes a sequence that is at least partially complementary to the rcTSO sequence. In some embodiments, the TSO primer includes a sequence that is 1, 2, 3, 4, 5, 6, 7, 8, 9, 10, 11, 12, 13, 14, 15, 16, 17, 18, 19, 20, 21, 22, 23, 24, 25, or more nucleotides in length. In some embodiments, the TSO primer includes any of the deoxyribonucleic acids, ribonucleic acids, modified nucleic acids, or any combination therein (e.g., any of the nucleotide derivatives or combinations thereof described herein). In some embodiments, the TSO primer includes RNA bases. In some embodiments, the TSO primer does not include RNA bases.

In some embodiments, the TSO primer is a single-stranded nucleic acid where the 3′ end is used as a chemical substrate for a nucleic acid polymerase in a nucleic acid extension reaction. In some embodiments, the TSO primer is used as a chemical substrate for a second strand synthesis where the extended capture probe is used as a template in a nucleic acid extension reaction, where the second strand is complementary to all or a portion of the cDNA molecule and all or a portion of the capture probe. In some embodiments, the TSO primer and a first enzyme (e.g., a reverse transcriptase with DNA polymerase functionality) are used in second strand synthesis, where the second strand synthesis occurs in the same reaction as the reverse transcription. In some embodiments, the TSO primer and a second enzyme (e.g., a DNA polymerase) are used in second strand synthesis reaction, where the second strand synthesis occurs in the same reaction as the reverse transcription.

In some embodiments, the method includes contacting the nucleic acid bound to the capture probe with a composition that includes a TSO blocking moiety. In some embodiments, the TSO blocking moiety is a nucleotide sequence that is 1, 2, 3, 4, 5, 6, 7, 8, 9, 10, 11, 12, 13, 14, 15, 16, 17, 18, 19, 20, 21, 22, 23, 24, 25, or more nucleotides in length. In some embodiments, the TSO blocking moiety is a nucleotide sequence that is at least partially complementary to the TSO. In some embodiments, the TSO blocking moiety prohibits the TSO primer from interacting with the rcTSO. For example, the TSO blocking moiety can bind to the TSO primer thereby inhibiting the TSO primer from interacting with the rcTSO. In some embodiments, the TSO blocking moiety is a nucleotide sequence that is at least partially complementary to the rcTSO. In some embodiments, the TSO blocking moiety prohibits the rcTSO from interacting with the TSO primer. For example, the TSO blocking moiety can bind to the rcTSO thereby inhibiting the rcTSO from interacting with the TSO primer.

(e) Biological Sample, Analytes and Sample Preparation

1. Biological Samples and Analytes

Methods disclosed herein can be performed on any type of sample. In some embodiments, the sample is a fresh tissue sample. In some instances, the biological sample is a tissue, a tissue section, an organ, an organism, or a cell culture sample. In some instances, the biological sample is a formalin-fixed, paraffin-embedded (FFPE) sample, a frozen sample, or a fresh sample.

In some embodiments, the analyte includes one or more of RNA, DNA, a protein, a small molecule, and a metabolite. In some embodiments, the analyte (e.g., target analyte) is a single-stranded oligonucleotide. In some embodiments, the single-stranded oligonucleotide is RNA. In some embodiments, the RNA is mRNA. In some embodiments, the mRNA is an mRNA of interest. In some embodiments, the multiple target analytes are detected. The multiple targets can, in some instances, include sequences that have at least 80%, at least 85%, at least 90%, at least 91%, at least 92%, at least 93%, at least 94%, at least 95%, at least 96%, at least 97%, at least 98%, or at least 99% sequence identity to each other. In some instances, the multiple targets each include one or more conserved sequences. In some instances, the multiple targets are mRNAs that encode for proteins that have a similar function. In some instances, the multiple targets are mRNAs that encode for proteins that function in the same or a similar cellular pathway.

In some embodiments, methods provided herein include identifying a location of one or more analytes where one or more analytes refers to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, about 500, about 600, about 700,about 800,about 900, about 1,000 analytes, about 2,000 analytes, about 3,000 analytes, about 4,000 analytes, about 5,000 analytes, about 6,000 analytes, about 7,000 analytes, about 8,000 analytes, about 9,000 analytes, about 10,000 analytes, about 50,000 analytes, about 100,000 analytes, about 200,000 analytes, about 500,000 analytes, or about 1,000,000 analytes.

Subjects from which biological samples can be obtained can be healthy or asymptomatic individuals that have or are suspected of having a disease (e.g., cancer) or a pre-disposition to a disease, and/or individuals that are in need of therapy or suspected of needing therapy. In some instances, the biological sample can include one or more diseased cells. A diseased cell can have altered metabolic properties, gene expression, protein expression, and/or morphologic features. Examples of diseases include inflammatory disorders, metabolic disorders, nervous system disorders, and cancer. In some instances, the biological sample includes cancer or tumor cells. Cancer cells can be derived from solid tumors, hematological malignancies, cell lines, or obtained as circulating tumor cells. In some instances, the biological sample is a heterogenous sample. In some instances, the biological sample is a heterogenous sample that includes tumor or cancer cells and/or stromal cells.

In some embodiments, the biological sample is from a human subject.

FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of poly(T) capture and reverse transcription of mRNA). In certain embodiments, methods provided herein enable sensitive measurement of specific genes of interest that otherwise might be missed with a whole transcriptomic approach.

In some instances, FFPE samples are stained (e.g., using H&E, immunofluorescence, etc.). The methods disclosed herein are compatible with staining methods that will allow for morphological context overlaid with transcriptomic analysis. However, depending on the need some samples may be stained with only a nuclear stain, such as staining a sample with only hematoxylin and not eosin, the use of DAPI, etc. when location of a cell nucleus is needed.

In some embodiments, a biological sample (e.g. tissue section) can be fixed with methanol, stained with hematoxylin and eosin, and imaged. In some instances, the biological sample is fixed before adding the probes to the biological sample. In some instances, the biological sample is fixed after adding the probes to the biological sample.

In some embodiments, fixing, staining, and imaging occurs before one or more probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the substrate.

In some embodiments, the FFPE sample is deparaffinized, permeabilized, equilibrated, and blocked before target probe oligonucleotides are added. In some embodiments, deparaffinization includes the use of xylenes. In some embodiments, deparaffinization includes multiple washes with xylenes. In some embodiments, deparaffinization includes multiple washes with xylenes followed by removal of xylenes using multiple rounds of graded alcohol washes followed by washing the sample with water. In some aspects, the water is deionized water. In some embodiments, equilibrating and blocking includes incubating the sample in a pre-Hyb buffer. In some embodiments, the pre-Hyb buffer includes yeast tRNA. In some embodiments, permeabilizing a sample includes washing the sample with a phosphate buffer. In some embodiments, the buffer is PBS. In some embodiments, the buffer is PBST.

As used herein, the term “spatially-tagged” or “spatially-tagged biological sample” may refer to a biological sample that has been exposed to a first capture probe, a second capture probe or both.

2. Imaging and Staining

In some instances, biological samples can be stained using a wide variety of stains and staining techniques. In some instances, the biological sample is stained before adding the probes to the biological sample. In some instances, the biological sample is stained after adding the probes to the biological sample.

In some instances, the biological sample is a section of a tissue (e.g., a 10 μm section). In some instances, the biological sample is dried after placement onto a glass slide. In some instances, the biological sample is dried at 42° C. In some instances, drying occurs for about 1 hour, about 2, hours, about 3 hours, or until the sections become transparent. In some instances, the biological sample can be dried overnight (e.g., in a desiccator at room temperature).

In some embodiments, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample. In some instances, imaging the sample occurs prior to deaminating the biological sample. In some instances, the sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some instances, the stain is an H&E stain.

In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, biological samples can be destained. Methods of destaining or decoloring a biological sample are known in the art, and generally depend on the nature of the stain(s) applied to the sample. For example, H&E staining can be destained by washing the sample in HCl, or any other acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in an acid (e.g., HCl). In some embodiments, destaining can include adding HCl to a downstream solution (e.g., permeabilization solution). In some embodiments, destaining can include dissolving an enzyme used in the disclosed methods (e.g., pepsin) in an acid (e.g., HCl) solution. In some embodiments, after destaining hematoxylin with an acid, other reagents can be added to the destaining solution to raise the pH for use in other applications. For example, SDS can be added to an acid destaining solution in order to raise the pH as compared to the acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., capture probe array), wherein analytes (e.g., proteins) are probed using immunofluorescence protocols. For example, the sample can be rehydrated, blocked, and permeabilized (3×SSC, 2% BSA, 0.1% Triton X, 1 U/μl RNAse inhibitor for 10 minutes at 4° C.) before being stained with fluorescent primary antibodies (1:100 in 3×SSC, 2% BSA, 0.1% Triton X, 1 U/μl RNAse inhibitor for 30 minutes at 4° C.). The biological sample can be washed, coverslipped (in glycerol+1 U/μl RNAse inhibitor), imaged (e.g., using a confocal microscope or other apparatus capable of fluorescent detection), washed, and processed according to analyte capture or spatial workflows described herein.

In some instances, a glycerol solution and a cover slip can be added to the sample. In some instances, the glycerol solution can include a counterstain (e.g., DAPI).

As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some embodiments, optimizing permeabilization can be useful for identifying intracellular analytes. Permeabilization optimization can include selection of permeabilization agents, concentration of permeabilization agents, and permeabilization duration. Tissue permeabilization is discussed elsewhere herein.

In some embodiments, blocking an array and/or a biological sample in preparation of labeling the biological sample decreases nonspecific binding of the antibodies to the array and/or biological sample (decreases background). Some embodiments provide for blocking buffers/blocking solutions that can be applied before and/or during application of the label, wherein the blocking buffer can include a blocking agent, and optionally a surfactant and/or a salt solution. In some embodiments, a blocking agent can be bovine serum albumin (BSA), serum, gelatin (e.g., fish gelatin), milk (e.g., non-fat dry milk), casein, polyethylene glycol (PEG), polyvinyl alcohol (PVA), or polyvinylpyrrolidone (PVP), biotin blocking reagent, a peroxidase blocking reagent, levamisole, Camoy's solution, glycine, lysine, sodium borohydride, pontamine sky blue, Sudan Black, trypan blue, FITC blocking agent, and/or acetic acid. The blocking buffer/blocking solution can be applied to the array and/or biological sample prior to and/or during labeling (e.g., application of fluorophore-conjugated antibodies) to the biological sample.

3. Preparation of Sample for Application of Probes

In some instances, additional reagents are added to the biological sample, prior to the addition of the probes. Additional reagents can be any reagent known in the art, so long as it preserves the integrity of an analyte.

In some instances, the biological sample is deparaffinized. Deparaffinization can be achieved using any method known in the art. For example, in some instances, the biological sample is treated with a series of washes that include xylene and various concentrations of ethanol. In some instances, methods of deparaffinization include treatment with xylene (e.g., three washes at 5 minutes each). In some instances, the methods further include treatment with ethanol (e.g., 100% ethanol, two washes 10 minutes each; 95% ethanol, two washes 10 minutes each; 70% ethanol, two washes 10 minutes each; 50% ethanol, two washes 10 minutes each). In some instances, after ethanol washes, the biological sample can be washed with deionized water (e.g., two washes for 5 minutes each). It is appreciated that one skilled in the art can adjust these methods to optimize deparaffinization.

In some instances, the biological sample is decrosslinked. In some instances, the biological sample is decrosslinked in a solution containing TE buffer (comprising Tris and EDTA). In some instances, the TE buffer is basic (e.g., at a pH of about 9). In some instances, decrosslinking occurs at about 50° C. to about 80° C. In some instances, decrosslinking occurs at about 70° C. In some instances, decrosslinking occurs for about 1 hour at 70° C. Just prior to decrosslinking, the biological sample can be treated with an acid (e.g., 0.1M HCl for about 1 minute). After the decrosslinking step, the biological sample can be washed (e.g., with 1×PBST).

In some instances, the methods of preparing a biological sample for probe application include permeabilizing the sample. In some instances, the biological sample is permeabilized using a phosphate buffer. In some instances, the phosphate buffer is PBS (e.g., 1×PBS). In some instances, the phosphate buffer is PBST (e.g., 1×PBST). In some instances, the permeabilization step is performed multiple times (e.g., 3 times at 5 minutes each).

In some embodiments of any of the methods for identifying a location of an analyte in a biological sample, the method includes permeabilizing (e.g., using any of the exemplary permeabilization methods described herein) the biological sample before, contemporaneously with, or after exposing the biological sample to the capture probe. In some embodiments of any of the methods for identifying a location of an analyte in a biological sample, the method includes processing the biological sample using any of the methods described herein. For example, the method can include selecting a region of interest of the biological sample by laser-capture microdissection prior to exposing the biological sample to the capture probe.

In some instances, the methods of preparing a biological sample for probe application include steps of equilibrating and blocking the biological sample. In some instances, equilibrating is performed using a pre-hybridization (pre-Hyb) buffer. In some instances, the pre-Hyb buffer is RNase-free. In some instances, the pre-Hyb buffer contains no bovine serum albumin (BSA), solutions like Denhardt's, or other potentially nuclease-contaminated biological materials.

In some instances, the equilibrating step is performed multiple times (e.g., 2 times at 5 minutes each; 3 times at 5 minutes each). In some instances, the biological sample is blocked with a blocking buffer. In some instances, the blocking buffer includes a carrier such as tRNA, for example yeast tRNA (e.g., at a final concentration of 10-20 μg/mL). In some instances, blocking can be performed for 5, 10, 15, 20, 25, or 30 minutes.

Any of the foregoing steps can be optimized for performance. For example, one can vary the temperature. In some instances, the pre-hybridization methods are performed at room temperature. In some instances, the pre-hybridization methods are performed at 4° C. (in some instances, varying the timeframes provided herein).

(f) Kits

In some embodiments, also provided herein are kits that include one or more reagents to detect one or more analytes described herein, wherein one or more steps is performed at the same time. In some instances, the kit includes a plurality of capture probes on a subsrate. In some instances, each capture probe includes a capture domain.

In some instances, kits further include one or more of a buffer to perform reverse transcription and second strand synthesis; a reverse transcriptase; a polymerase; a template switching oligonucleotide (TSO); a sequence complementary to a TSO; dNTPs. In some instances, the kit includes instructions to perform any of the methods disclosed herein.

A non-limiting example of a kit used to perform any of the methods described herein includes: (a) a substrate comprises a plurality of captures probes; (b) reagents (e.g., buffers, dNTPs, TSO and sequences complementary to TSOs), (c) one or more enzymes (e.g., a reverse transcriptase and a polymerase); and (d) instructions for performing the methods provided herein.

### EXAMPLES

### Example 1: Exemplary Method for Processing a Nucleic Acid in a Biological Sample by Performing Reverse Transcription and Second Strand Synthesis in One Reaction

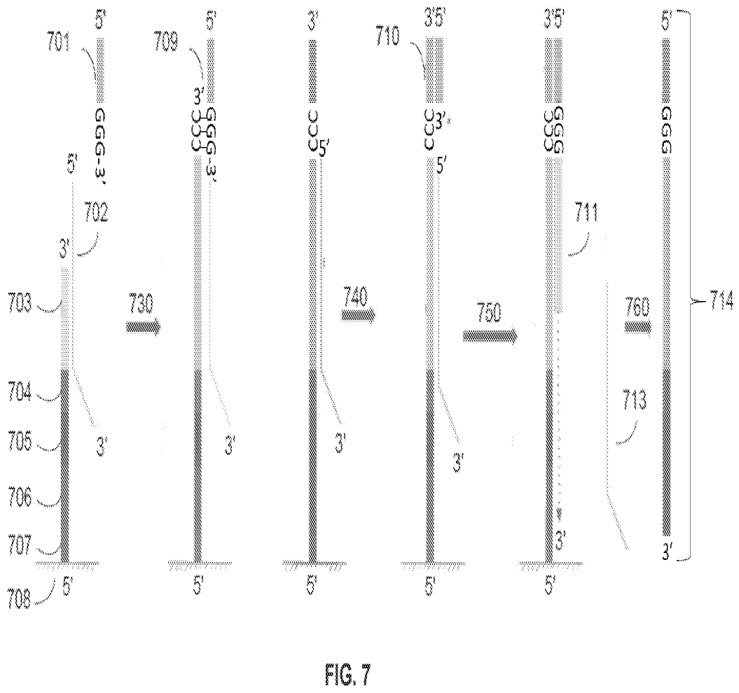

As seen in FIG. 7, a non-limiting exemplary method for processing a nucleic acid in a biological sample can include a single reaction where a first enzyme is used for both first strand synthesis and second strand synthesis. The method includes a spatial array 708 with a plurality of capture probes thereon. On the spatial array, a capture probe is affixed to the surface of the spatial array. The capture probe includes a constant sequence 707, a read 1 sequence 706, a spatial barcode 705, a UMI 704, and a capture domain 703. The array is contacted with a biological sample in a manner that enables the capture probes to capture an analyte 702 from the biological sample. After capture, the array is exposed to a composition that includes a buffer, a reverse transcriptase enzyme, a reverse complement of the TSO (rcTSO) 710, and a TSO primer 701. In steps 730, 740, and 750, the reverse transcriptase enzyme reverse transcribes the captured mRNA to generate a complementary DNA (cDNA) molecule 711. The terminal transferase activity 709 of reverse transcriptase adds un-templated cytosines to the 3′ end of the cDNA molecule. The template switching oligonucleotide (TSO) includes a hybridization region and a template region (e.g., TSO primer region) in step 730. The hybridization region includes a sequence that is at least partially complementary to the un-templated cytosines. Thus, the hybridization region binds the TSO to the cytosines on the 3′ of the cDNA molecule. Upon binding, in step 740, the reverse transcriptase transcribes the TSO thereby adding the complementary sequence of the TSO (rcTSO) to the 3′ end of the cDNA molecule.

In step 750, the TSO primer binds to the rcTSO sequence. In addition to reverse transcriptase functionality, the reverse transcriptase also includes DNA polymerase activity and RNA strand displacement activity. The same reverse transcriptase enzyme used for first strand cDNA synthesis is used to (1) displace the mRNA 713 adhered to the cDNA and (2) generate a second strand. The processivity of the DNA polymerase activity is tied to the ability of the reverse transcriptase to remove the mRNA (e.g., the RNA strand displacement). Utilizing the gap (unpaired nucleotides) between the TSO and the mRNA still adhered to the cDNA, the strand displacement activity of the reverse transcriptase displaces the mRNA. Once a full length second strand 714 is generated in step 760, the second strand is released from the extended capture probe and is ready for further downstream processing.

### Example 2: Method for Identifying the Spatial Location of a Nucleic Acid in a Biological Sample

A set of experiments was performed to assess a method for identifying the spatial location of a nucleic acid in a biological sample by combining reverse transcription (first strand synthesis of complementary DNA (cDNA)) and second strand synthesis into a single reaction. The method for identifying the spatial location of a nucleic acid combining reverse transcription and second strand synthesis was compared to the method for identifying the spatial location of a nucleic acid where reverse transcription and second strand synthesis were performed in two separate reactions. Mouse brain sections were used as the biological sample.

The biological sample was processed according to a method described herein. Briefly, the biological sample was exposed to a permeabilization enzyme and incubated at 37° C. for the pre-determined permeabilization time. The permeabilization enzyme was removed and the sample prepared for analyte capture by adding 0.1×SSC buffer. Analyte capture was then performed by contacting the biological sample with a spatial array in a manner that enables capture of the nucleic acids by the capture probes on the spatial array.

A master mix was added to the samples on the spatial array (i.e., the spatial array that included nucleic acids bound to the capture probes). The master mix added to the spatial array included one of three different compositions: (composition 1) (“combined”) containing nuclease-free water, a reverse transcriptase reagent, a TSO, a reducing agent, a M-MLV reverse transcriptase enzyme, and a TSO primer; (composition 2) (“combined+bst”) containing nuclease-free water, a reverse transcriptase reagent, a TSO, a reducing agent, a M-MLV reverse transcriptase enzyme, a TSO primer, and a Bst DNA polymerase; or (composition 3) (“combined+bst+smPCR2) containing nuclease-free water, a reverse transcriptase reagent, a TSO, a reducing agent, a M-MLV reverse transcriptase enzyme, a TSO primer, a Bst DNA polymerase, and an additional TSO primer (“smPCR2”). A control reaction (“separate (control)”) was included where composition 1 was added to the spatial array but where a separate second reaction was used to perform the second strand synthesis.

The samples were exposed to two different thermocycling protocols. The first thermocycling protocol included a first incubation at 53° C. for 45 minutes and a second incubation at 65° C. for 2 hours. The second thermocycling protocol included a single incubation at 53° C. for 2 hours. After completing the thermocycling, the samples were processed for further downstream analysis (e.g., amplification and library preparation according to the method described herein). For the control sample, following reverse transcription, physical denaturation and washing, a second strand mix, including a second strand reagent, a second strand primer, and a second strand enzyme, was added to the sample and the sample was sealed and incubated. At the end of the incubation, the reagents were removed and elution buffer was added and removed from the sample, and 0.8M KOH was added again to the sample and the sample was incubated for 10 minutes at room temperature. Tris-HCl was added and the reagents were mixed. The control sample was transferred to a new tube, vortexed, and placed on ice.

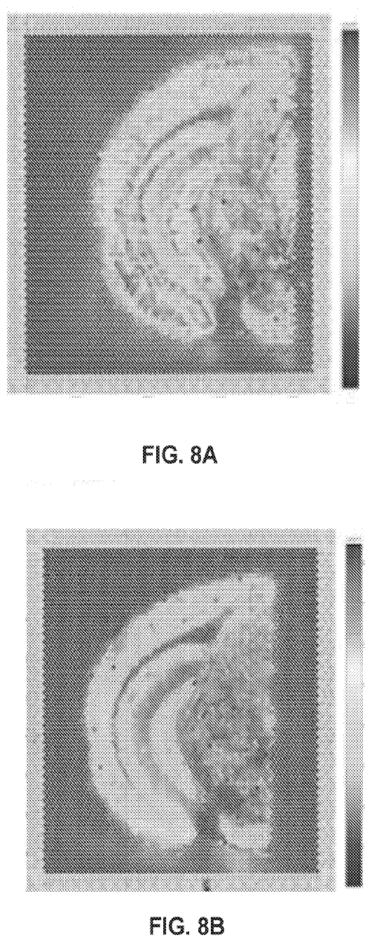

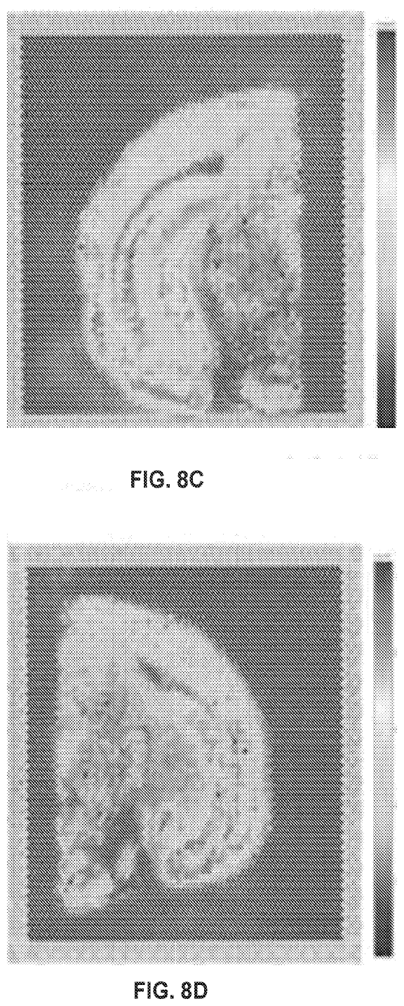

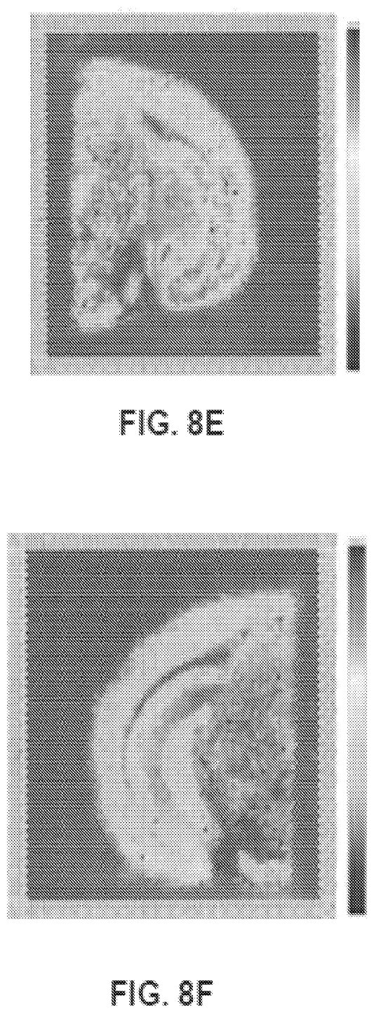

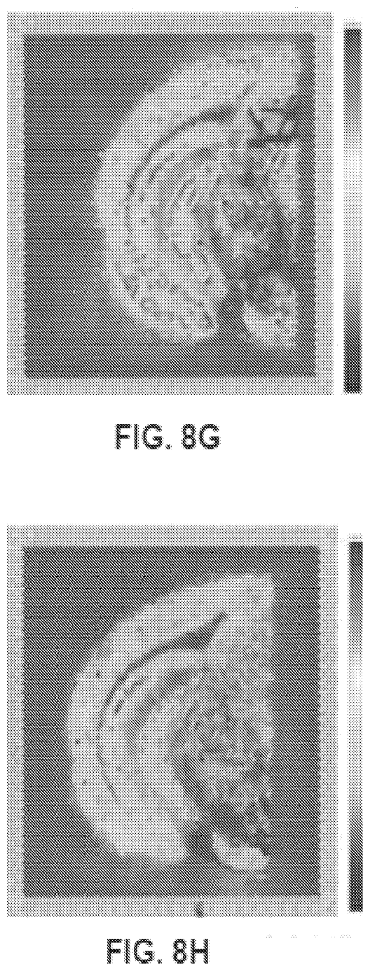

The biological samples were processed by following the exemplary steps for cDNA amplification and quality control as described herein. In addition, the biological sample was processed by following the exemplary steps for spatial gene expression library construction. The library was sequenced using available Illumina sequencing platforms. Spatially-resolved gene expression in the mouse brain is shown in FIGS. 8A-8H as a heat map of transcripts detected at each spot on the spatial array. FIGS. 8A-8B show heat maps of transcripts for samples treated with composition 1 (“combined”) and following incubation with the first and second thermocyling protocols, respectively. FIGS. 8C-8D show heat maps of transcripts for samples treated with composition 2 (“combined+bst) and following incubation with the first and second thermocyling protocols, respectively. FIGS. 8E-8F show heat maps of transcripts for samples treated with composition 3 (”combined+bst+smPCR2) and following incubation with the first and second thermocyling protocols, respectively. FIGS. 8G-8H show heat maps of transcripts for separate (control) sample (i.e., separate reactions for reverse transcription and second strand synthesis) following incubation with the first and second thermocyling protocols, respectively, for the reverse transcription reaction.

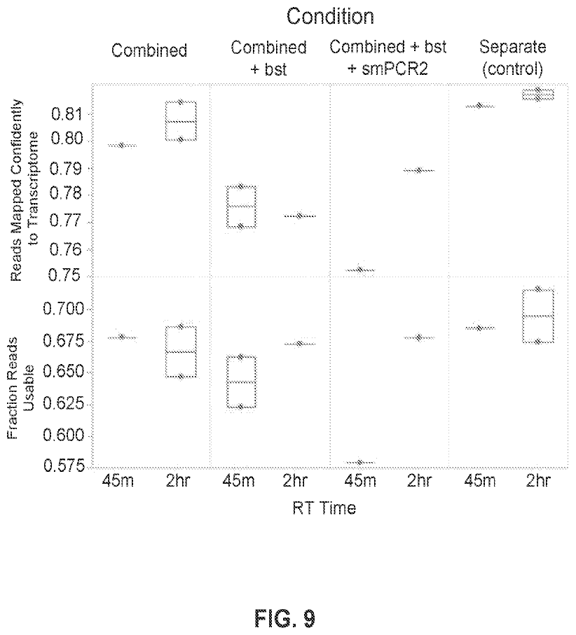

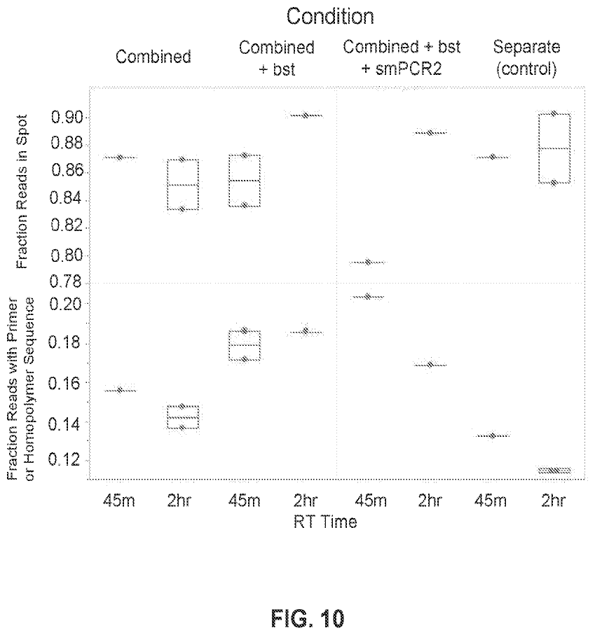

In FIG. 9, assessment of the mapped reads and usable reads generated by each of the conditions shown in the plots in FIGS. 8A-8H shows the “combined” sample performed comparably to the separate (control) sample. In addition, in FIG. 10, assessment of the type of reads within each spot for each of the conditions shown in the plots in FIGS. 8A-8H shows that the type of reads are comparable across each condition. Here, the type of read (e.g., primer, homopolymer) is an indicator of poor quality reads.

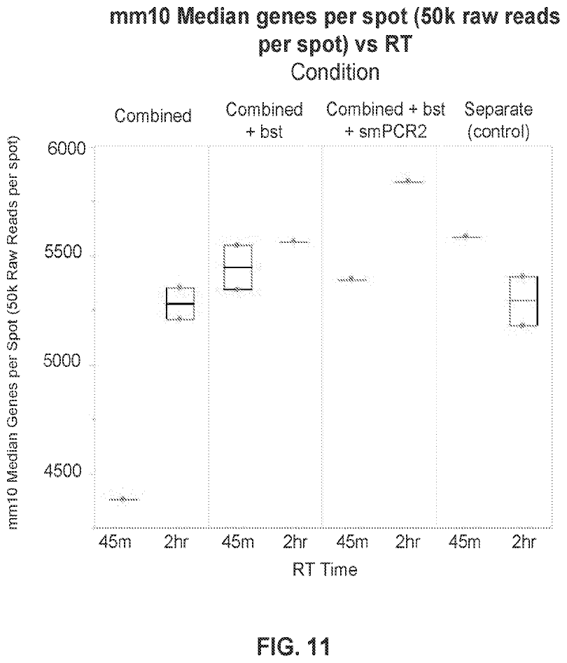

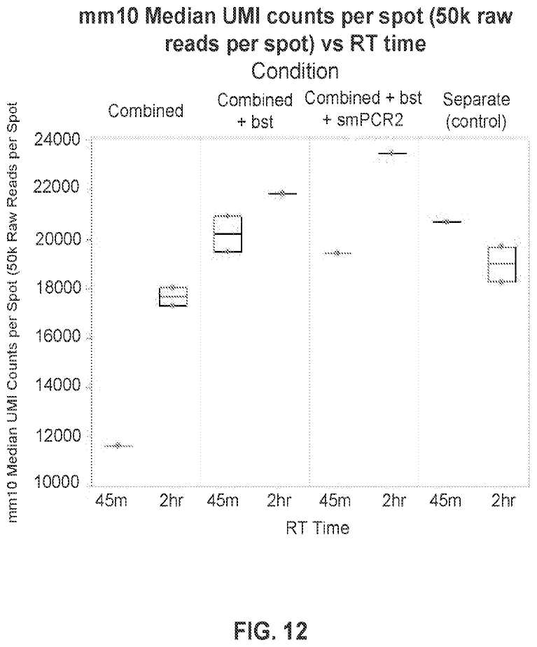

For both median genes per spot and median UMIs per spot, the combined+bst and combined+bst+smPCR2 samples performed comparably to the separate (control). FIG. 11 shows median genes per spot for each of the plots in FIGS. 8A-8H. FIG. 12 shows median UMIs per spot for each of the plots in FIGS. 8A-8H.

Taken together, the data demonstrate that the steps of reverse transcriptase and second strand synthesis can be combined in one reaction without loss of analyte detection sensitivity.

### Example 3: Additional Method for Identifying the Spatial Location of a Nucleic Acid in a Biological Sample

A second set of experiments was performed to assess a method for identifying the spatial location of a nucleic acid in a biological sample by combining reverse transcription (first strand synthesis of complementary DNA (cDNA)) and second strand synthesis into a single reaction. The method for identifying the spatial location of a nucleic acid combining reverse transcription and second strand synthesis was compared to the method for identifying the spatial location of a nucleic acid where reverse transcription and second strand synthesis were performed in two separate reactions. Mouse brain sections were used as the biological sample.

The biological sample was processed as described herein. Briefly, the biological sample was exposed to a permeabilization enzyme and incubated at 37° C. for the pre-determined permeabilization time. The permeabilization enzyme was removed and the sample prepared for analyte capture by adding 0.1×SSC buffer. Analyte capture was then performed by contacting the biological sample with a spatial array in a manner that enables capture of the nucleic acids by the capture probes on the spatial array.

Next, a master mix was added to the samples on the spatial array (i.e., the spatial array that included nucleic acids bound to the capture probes). The master mix added to the spatial array included two different conditions:

Condition 1: (“combined RT and SS”) one formulation is used in Condition 1. The one formulation comprises nuclease-free water, a reverse transcriptase reagent, a TSO, a reducing agent, a Bst DNA polymerase, and an M-MLV reverse transcriptase. In one reaction, the sample was mixed with the formulation and preheated to 53° C. Reverse transcription occurred for 10 minutes at 53° C. Then, the temperature was increased for 15 minutes at 65° C. for second strand synthesis to occur.

Condition 2: two separate reactions with two formulations (an RT formulation and a second strand synthesis formulation). The RT formulation comprises nuclease-free water, a reverse transcriptase reagent, a TSO, a reducing agent, and an M-MLV reverse transcriptase. For the reverse transcription reaction, the sample was heated to 53° C. for 45 minutes. Then the biological sample was purified and subjected to second strand (SS) synthesis. SS conditions included adding SS buffer, SS primer, and an SS enzyme (e.g., Kapa polymerase) to the sample, followed by heating the sample for 15 minutes at 65° C.

After the above reactions, the samples were stored at 4° C. until use.

The biological samples were processed by following the exemplary steps for cDNA amplification and quality control as described herein. In addition, the biological sample was processed by following the exemplary steps for spatial gene expression library construction. The library was sequenced using available Illumina sequencing platforms.

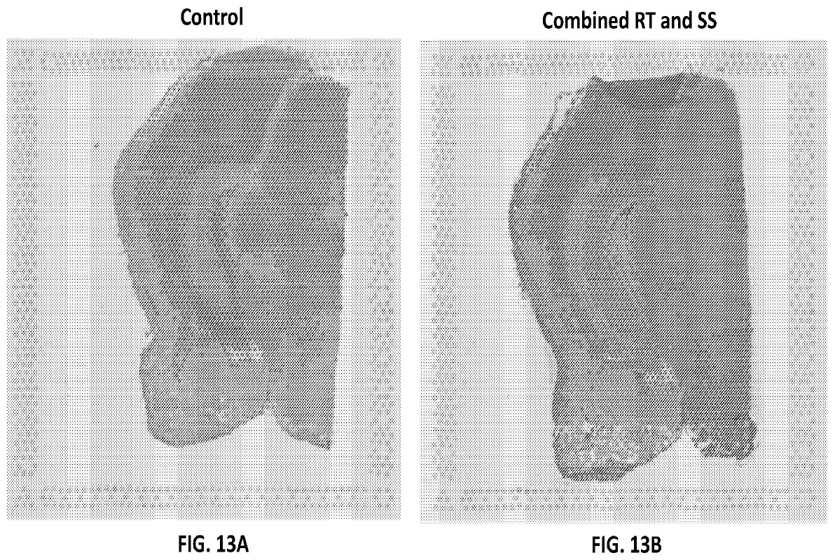

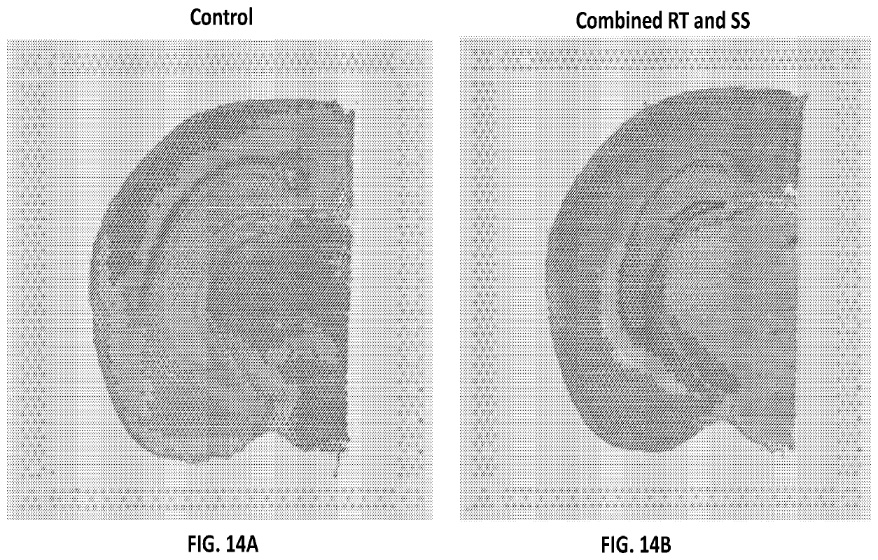

Spatially-resolved gene expression in the mouse brain is shown in FIGS. 13A-13B (brain sample 1) and FIGS. 14A-14B (brain sample 2) as a heat map of transcripts detected at each spot on the spatial array. FIGS. 13A-13B and FIGS. 14A-14B show heat maps of transcripts for samples treated with condition 1 (FIGS. 13B and 14B) or with condition 2 (FIGS. 13A and 14A).

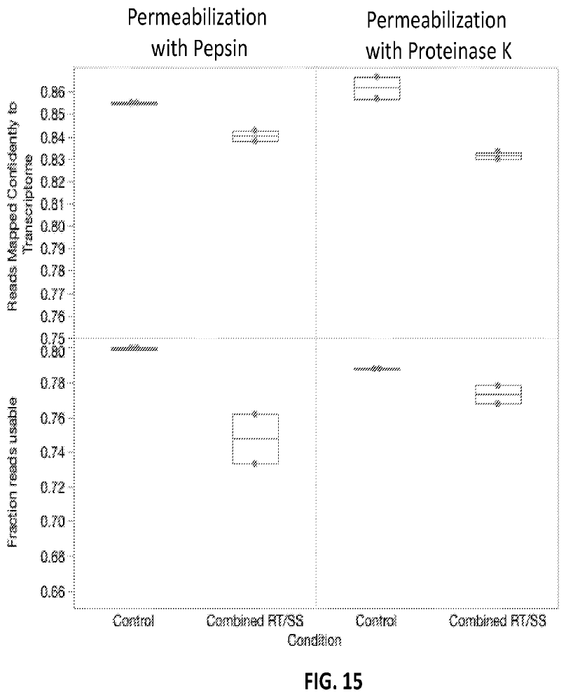

In FIG. 15, assessment of the mapping reads and usable reads generated by each of the conditions shown in the plots in FIGS. 13A-13B and FIGS. 14A-14B, shows the “combined” sample performed comparably to the separate (control) sample under conditions in which the biological samples were permeabilized with pepsin or with proteinase K.

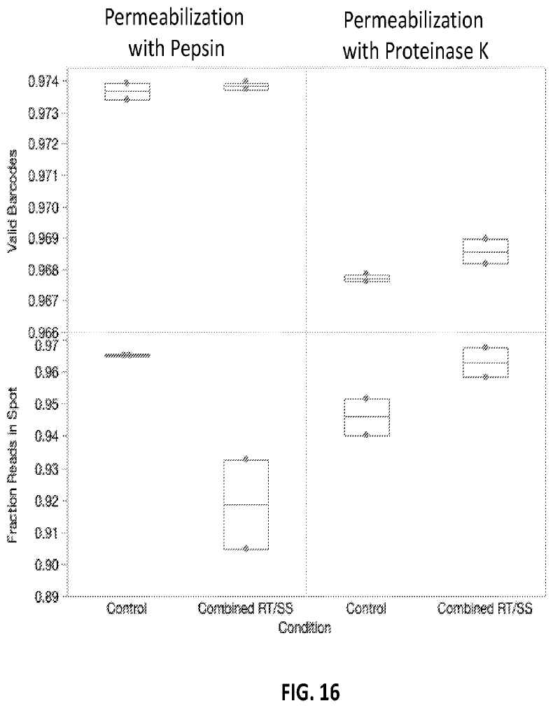

In addition, assessment of valid barcodes and fractions of reads per spot was performed under conditions in which the biological samples were permeabilized with pepsin or with proteinase K. As shown in FIG. 16, in either condition, the number of valid barcodes was similar whether Condition 1 or Condition 2 was used. Further, the fraction of reads for each condition under either permeabilization settings resulted in fraction reads per spot above 0.9.

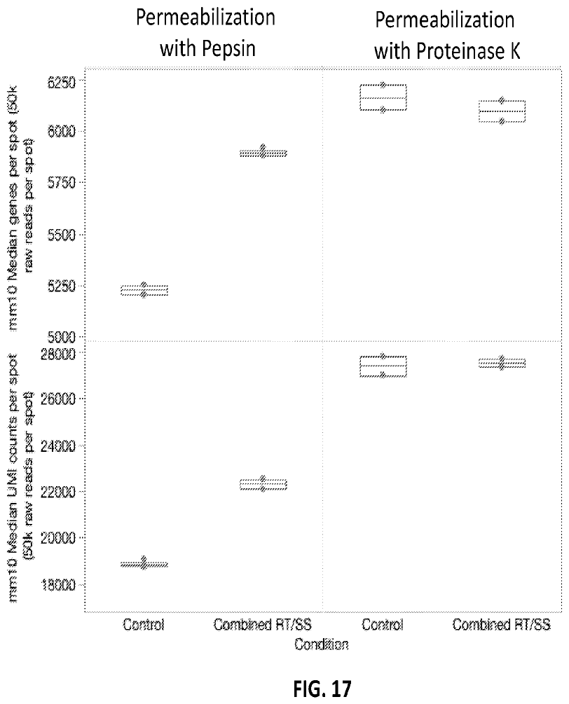

For both median genes per spot and median UMIs per spot in the brain samples shown in FIGS. 13A-13B and FIGS. 14A-14B, the combined RT and SS condition (Condition 2) performed comparably to the separate (control) (Condition 1). FIG. 17 shows median genes per spot and median UMIs per spot for each of the plots in FIGS. 13A-13B and FIGS. 14A-14B.

Taken together, the data demonstrate that the steps of reverse transcriptase and second strand synthesis can be combined in one reaction without loss of analyte detection.

What is claimed is:

1. A method of determining abundance and location of an analyte in a biological sample comprising:
(a) contacting a biological sample with a substrate, wherein the substrate comprises a plurality of capture probes attached to the surface of the substrate, and wherein a capture probe of the plurality of capture probes comprises a spatial barcode and a capture domain;
(b) hybridizing the analyte to the capture probe;
(c) in a single reaction, generating a complementary DNA (cDNA) molecule of the analyte by reverse transcription and performing second strand synthesis of the cDNA molecule by contacting the analyte with a composition comprising a reverse transcription enzyme and a strand-displacing polymerase; and
(d) determining (i) all or a portion of the sequence of the spatial barcode or the complement thereof, and (ii) all or a portion of the sequence of the analyte, or a complement thereof; and using the determined sequences of (i) and (ii) to identify the abundance and location of the analyte in the biological sample.

2. The method of claim 1, wherein the composition further comprises a buffer and one or more reagents selected from a plurality of dNTPs, a plurality of template switching oligonucleotides (TSOs), and a plurality of sequences comprising a reverse complement of the TSO (rcTSO).

3. A method of processing an analyte from a biological sample, comprising:
(a) hybridizing the analyte from the biological sample to a capture probe, wherein the capture probe comprises a capture domain and a spatial barcode;
(b) contacting the analyte with a composition comprising:
(i) a buffer;
(ii) one or more enzymes selected from a reverse transcription enzyme and a strand-displacing polymerase; and
(iii) one or more reagents selected from a plurality of dNTPs, a plurality of template switching oligonucleotides (TSOs), and a plurality of sequences complementary to the TSOs; and
(c) performing reverse transcription and second strand synthesis in one reaction.

4. The method of any one of the preceding claims, wherein the reverse transcription enzyme comprises one or more of terminal transferase activity, template switching ability, strand displacement ability, or combinations thereof

5. The method of claim 4, wherein the reverse transcription enzyme comprises a Moloney Murine Leukemia Virus (M-MLV) reverse transcriptase enzyme.

6. The method of claim 4, wherein the reverse transcription enzyme comprises M-MLV reverse transcriptase enzyme 42B.

7. The method of any one of the preceding claims, wherein the strand-displacing polymerase is a phi29 DNA polymerase.

8. The method of any one of claims 1-6, wherein the strand-displacing polymerase is a Bst DNA polymerase.

9. The method of any one of the preceding claims, wherein the composition comprises a template switching oligonucleotide (TSO).

10. The method of claim 9, wherein the TSO is about 10 to 50 nucleotides in length.

11. The method of claim 9 or 10, wherein the TSO comprises DNA.

12. The method of any one of claims 9-11, wherein the TSO comprises a homopolymer guanine sequence that hybridizes to a homopolymer cytosine sequence on the capture probe.

13. The method of any one of claims 9-12, wherein the TSO comprises a sequence that hybridizes to the capture probe.

14. The method of any one of the preceding claims, wherein the reverse transcription comprises:
(i) coupling the TSO to a 3′ end of the analyte; and
(ii) extending the capture probe using the analyte as a template, thereby generating an extended capture probe comprising a sequence that is complementary to the analyte and the TSO.

15. The method of any one of the preceding claims, wherein the second strand synthesis comprises:
(i) hybridizing the TSO to the rcTSO; and
(ii) extending the TSO using the extended capture probe as a template, thereby generating a second strand, wherein the second strand is complementary to all or a portion of the analyte and all or a portion of the capture probe.

16. The method of claim 15, wherein the rcTSO comprises DNA.

17. The method of claim 15 or 16, further comprising ligating the rcTSO to the capture probe using a ligase.

18. The method of claim 17, wherein the ligase is a T4 RNA ligase (Rnl2), a splintR ligase, a single stranded DNA ligase, or a T4 DNA ligase.

19. The method of any one of claims 15-18, wherein the rcTSO comprises a pre-adenylated phosphate group at its 5′ end, and wherein the first probe comprises at least two ribonucleic acid bases at the 3′ end.

20. The method of any one of claims 17-19, wherein the ligating the rcTSO to the capture probe comprises ligating a 3′ end of the capture probe to a 5′ end of the rcTSO.

21. The method of any one of claims 17-20, wherein the ligase is selected from the group consisting of: thermostable 5′ AppDNA/RNA Ligase, truncated T4 RNA Ligase 2, truncated T4 RNA Ligase 2 K227Q, truncated T4 RNA Ligase 2 KQ, Chlorella Virus PBCV-1 DNA Ligase, or any combination thereof.

22. The method of any one of claims 15-21, wherein the TSO binds the rcTSO of the extended capture probe immediately after reverse transcription.

23. The method of any one of the preceding claims, wherein second strand synthesis is performed immediately after the reverse transcription.

24. The method of any one of the preceding claims, wherein the method does not comprise a wash step between reverse transcription and second strand synthesis.

25. The method of any one of the preceding claims, wherein the composition further comprises a TSO blocking moiety, wherein the TSO blocking moiety prohibits the rcTSO interacting with the TSO and is released from the TSO prior to the second strand synthesis.

26. The method of any one of the preceding claims, further comprising releasing the second strand.

27. The method of claim 26, wherein the releasing the second strand comprises physical denaturation or chemical denaturation.

28. The method of any one of the preceding claims, further comprising permeabilizing the biological sample with a permeabilization agent.

29. The method of claim 28, wherein the permeabilization agent is selected from an organic solvent, a detergent, and an enzyme, or a combination thereof.

30. The method of claim 28, wherein the permeabilization agent is selected from the group consisting of: an endopeptidase, a protease sodium dodecyl sulfate (SDS), polyethylene glycol tert-octylphenyl ether, polysorbate 80, and polysorbate 20, N-lauroylsarcosine sodium salt solution, saponin, Triton X-100™, and Tween-20™.

31. The method of claim 30, wherein the endopeptidase is pepsin.

32. The method of claim 30, wherein the endopeptidase is proteinase K.

33. The method of any one of the preceding claims, wherein the method further comprises, prior to step (a), fixing the biological sample.

34. The method of claim 33, wherein the step of fixing the biological sample is performed using one or both of methanol and acetone.

35. The method of any one of the preceding claims, wherein the capture probe further comprises one or more functional domains, a unique molecular identifier (UMI), a cleavage domain, and combinations thereof.

36. The method of any one of the preceding claims, wherein the biological sample comprises a tissue section.

37. The method of any one of the preceding claims, wherein the biological sample comprises a formalin-fixed, paraffin-embedded (FFPE) sample, a frozen sample, or a fresh sample.

38. The method of any one of the preceding claims, wherein the biological sample comprises an FFPE sample.

39. The method of claim 38, wherein the FFPE tissue sample is decrosslinked.

40. The method of any one of the preceding claims, wherein the biological sample is removed from the substrate after hybridization of the analyte to the capture probe.

41. The method of any one of the preceding claims, wherein the biological sample was previously stained.

42. The method of claim 41, wherein the biological sample was previously stained using immunofluorescence or immunohistochemistry.

43. The method of claim 41 or 42, wherein the biological sample was previously stained using hematoxylin and eosin.

44. The method of any one of the preceding claims, wherein the analyte comprises a nucleic acid.

45. The method of claim 44, wherein the nucleic acid is an RNA molecule.

46. The method of claim 45, wherein the RNA molecule is an mRNA molecule.

47. The method of any one the preceding claims, wherein the determining step comprises amplifying all or part of the analyte, thereby producing an amplifying product.

48. The method of claim 47, wherein the amplifying product comprises (i) all or part of the analyte, or a complement thereof, and (ii) all or a part of the spatial barcode, or a complement thereof

49. The method of any one the preceding claims, wherein the determining step comprises sequencing.

50. The method of claim 49, wherein the sequencing comprises in situ sequencing, Sanger sequencing methods, next-generation sequencing methods, and nanopore sequencing.

51. A kit comprising
(a) a substrate comprises a plurality of captures probes attached to the surface of the substrate, wherein a capture probe of the plurality of capture probes comprises a spatial barcode and a capture domain;
(b) one or more reagents selected from a buffer, a plurality of dNTPs, a plurality of template switching oligonucleotides (TSOs); a plurality of sequences complementary to the TSOs;
(c) one or more enzymes selected from a reverse transcriptase and a polymerase;
and (d) instructions for performing the methods of any one of claims 1-50.
